(19) United States
(12) Patent Application Publication
(10) Pub. No.: US 2010/0094208 A1
(43) Pub. Date: Apr. 15, 2010

(54) DISPENSER FOR ADHESIVE NEWTONIAN FLUID

(75) Inventor: LODOVICO BRANCHETTI, CAMAIORE (IT)

Correspondence Address: Pearne & Gordon LLP, 1801 East 9th Street, Suite 1200, Cleveland, OH, 44114-3108, US

(73) Assignee: GEM S.R.L., CAMAIORE (LUCCA) (IT)

(21) Appl. No.: 12/575,167

(22) Filed: Oct. 7, 2009

(30) Foreign Application Priority Data
Oct. 10, 2008 (IT) PR2008A000063

(51) Int. Cl. A61M 5/19; A61M 5/31

(52) U.S. Cl. 604/82; 604/181; 604/191; 604/218

(57) ABSTRACT

Dispenser (1) for adhesive Newtonian fluid, comprising:
a syringe (5) containing the adhesive fluid;
a plunger (23) for pushing the adhesive fluid out of the outlet spout of the syringe (5);
an inlet (27) for a propellant gas;
means (28) for mixing the adhesive fluid coming out from the syringe (5) and the propellant gas coming from said inlet (27) in order to obtain a mixture deliverable by atomization on a surface, said means (28) for mixing being shaped in such a way that the mixing of the adhesive fluid and the propellant gas takes place in proximity to an open end (14a) of a cannula or catheter (14) of the dispenser (1).

The present invention relates to a dispenser for adhesive Newtonian fluid.

As is well known, a fluid is defined as Newtonian if its viscosity is constant irrespective of the shear stress applied to it. In particular, the present invention finds application in the medical sector, in the dispensing of biologically compatible adhesive materials or glues. Such adhesives are usually applied on biological surfaces during open surgery or laparoscopic operations.

In a Newtonian fluid (i.e. medical cyanoacrilates monomers), the relationship between shear stress and strain rate is linear (the coefficient of linearity being the “viscosity”) and independent from other factors, with the exception of temperature. In a non-Newtoninan fluid, the relationship between shear stress and strain rate is non-linear and can even be time-dependent, thus a constant coefficient of viscosity cannot be defined. For example, fibrin is a non-Newtonian fluid, and more precisely a thixotropic fluid since viscosity decreases with duration of stress.

Current state-of-the-art surgical techniques (for example in general, vascular, thoracic and urological surgery) use adhesives of synthetic origin, e.g. cyanoacrylates. The latter are formed by condensing formaldehyde with cyanoacetic ester, such as n-butyl or 2-octyl. Cyanoacrylates are able to polymerize in contact with weak bases, e.g. water or tissue surfaces. Furthermore, such adhesives are well tolerated and have negligible side effects.

There are known techniques for applying cyanoacrylate adhesives or glues by means of portable “dropper” dispensers. The dispensers currently on the market dispense the glue drop by drop or in the form of a continuous flow through a cannula.

“Spray” dispensers capable of spraying the glue over broader areas compared to “dropper” dispensers are also known. The functioning of “spray” dispensers is based on the chemical suspension of individual adhesive agents in flows of gas separated in such a way as to originate an equal number of jets or sprays that mix together in the air or directly on the tissue surface.

For example, the portable dispenser described in U.S. Pat. No. 4,359,049 comprises two syringes for dispensing two non-Newtonian adhesive agents that are then mixed and dispensed through a needle. The adhesive agents are dispensed by injecting two pressurized gas flows. In this manner, the adhesives delivered can reach a surface located at a distance of approximately 20 cm from the needle and adhere thereto, forming a film. Said dispenser, however, does not present any applicative advantage for Newtonian fluids.

The principal disadvantage of the dispenser described in U.S. Pat. No. 4,359,049 resides in the fact that the mixing of the two adhesive agents takes place outside the syringes, directly in the needle, thus proving ineffective. In fact, the final mixture takes on a non-uniform consistency and consequently provides an insufficient adhesive and cohesive strength for use in surgical operations.

Moreover, the gases employed are those generally present in an operating room, such as CO₂. Such gases manifest strong pressure fluctuations which introduce a significant non-uniformity into the sprays.

Furthermore, another disadvantage of the dispenser described is tied to the excessive rapidity of polymerization of the adhesive compounds on the impacted surfaces. In fact, the polymerization times typically range between about 5 and 20 seconds. Due to this characteristic (which in itself would be advantageous for surgical operations), the dispensing cannula becomes easily clogged and the quality of subsequent sprays is impaired.

Another solution, presented in U.S. Pat. No. 5,116,315, illustrates a “spray” dispenser for two adhesive agents provided with spray orifices that can be directly replaced by the user in the event they become clogged. However, the replacement of orifices entails an interruption in the surgical operation underway and undesired wastes of time. Moreover, the replacement may prove difficult for the user.

The document U.S. Pat. No. 6,432,084 describes another “spray” dispensing method. However, this solution exclusively regards non-Newtonian fluids. In particular, said solution employs a type of tip in which the adhesive agents are mixed prior to their impact with the carrier gases. This prevents the compound from polymerizing and obstructing the cannula before being delivered.

However, this solution, too, presents considerable disadvantages. The principal disadvantage resides in the use of gases present in the operating room as propellant gases for the adhesives. As already explained above, such gases manifest strong pressure fluctuations that introduce a significant non-uniformity into the sprays. Consequently, these dispensers do not allow fine deposits of adhesives to be achieved.

A further solution, disclosed in U.S. Pat. No. 5,154,320, describes the use of cyanoacrylates enclosed in an “aerosol”-type protective glass container.

However, the device described is not capable of delivering fluids at distances compatible with laparoscopic surgery.

Moreover, in this case as well, the outlet orifice is subject to frequent clogging.

Furthermore, since it is not possible to sterilize the external packaging of the dispenser, it cannot be used as a class III medical device.

Another disadvantage of this solution is tied to the impossibility of controlling the quantity of fluid delivered.

In this context, the technical task at the basis of the present invention is to propose a dispenser for adhesive Newtonian fluid which overcomes the limitations of the above-mentioned prior art.

In particular, it is an object of the present invention to provide a dispenser for adhesive Newtonian fluid such that the fluid dispensed polymerizes to form a thin, uniform film over the target surface.

Another object of the present invention is to propose a dispenser for adhesive Newtonian fluid such that the fluid dispensing cannula or nozzle is not clogged by the polymerization of the fluid.

A further object of the present invention is to provide a dispenser for adhesive Newtonian fluid in which it is possible to control both the quantity of fluid delivered and the area covered thereby.

A further object of the present invention is to provide a dispenser for adhesive Newtonian fluid having a simplified structure compared to the devices existing on the market.

A further object of the present invention is to provide a dispenser for adhesive Newtonian fluid that is easy to use.

Another object of the present invention is to propose a dispenser for adhesive Newtonian fluid usable in the medical sector, for example in open surgery or laparoscopic operations.

The defined technical task and the specified objects hereof are substantially achieved by a dispenser for adhesive Newtonian fluid comprising the technical characteristics described in one or more of the appended claims.

Further characteristics and advantages of the present invention will become more apparent from the following approximate, and hence non-restrictive, description of a preferred, but not exclusive, embodiment of a dispenser for adhesive Newtonian fluid as illustrated in the appended drawings, in which:

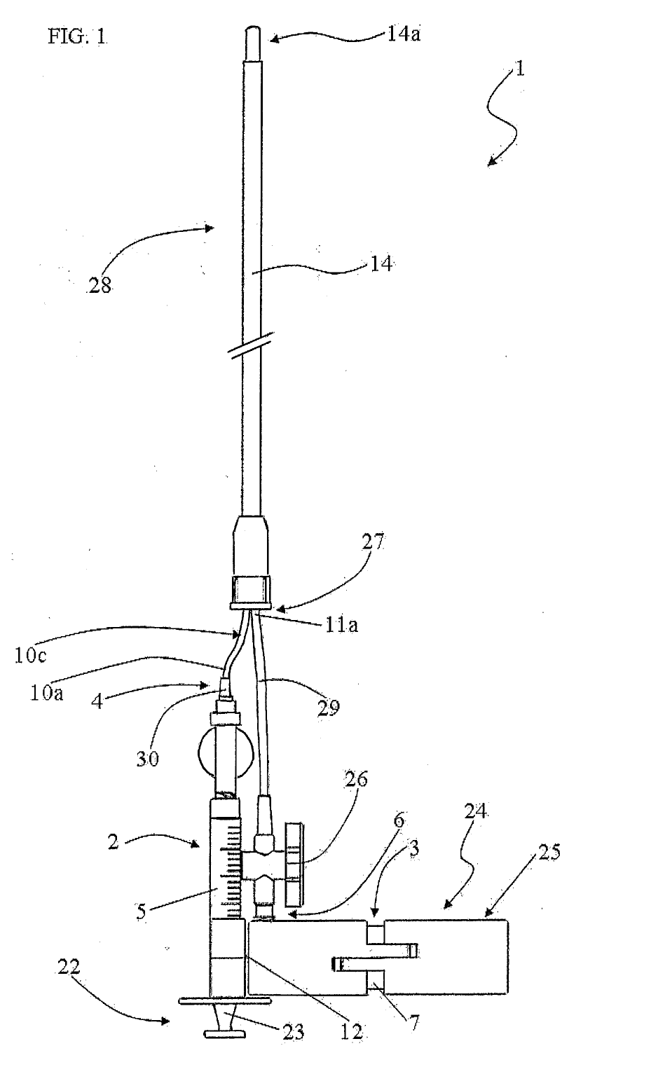

FIG. 1 illustrates a lateral view of a dispenser for adhesive Newtonian fluid, according to the present invention;

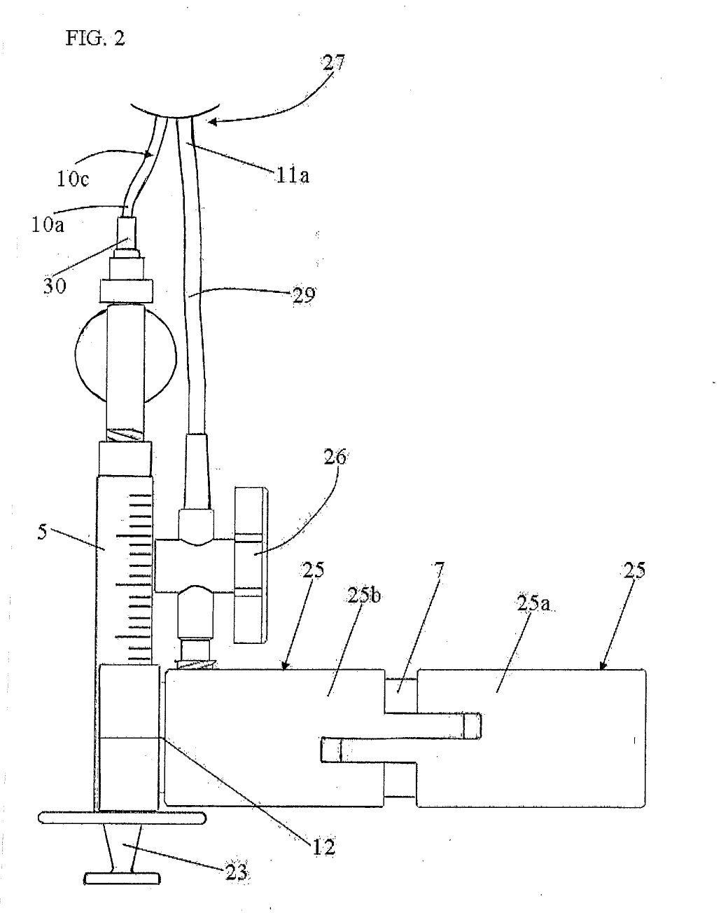

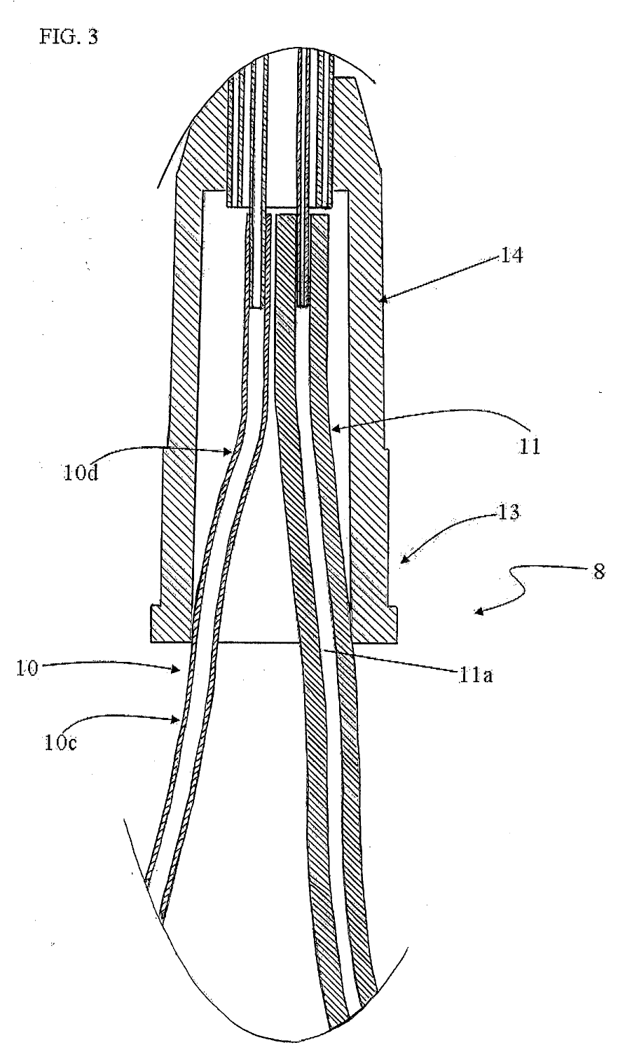

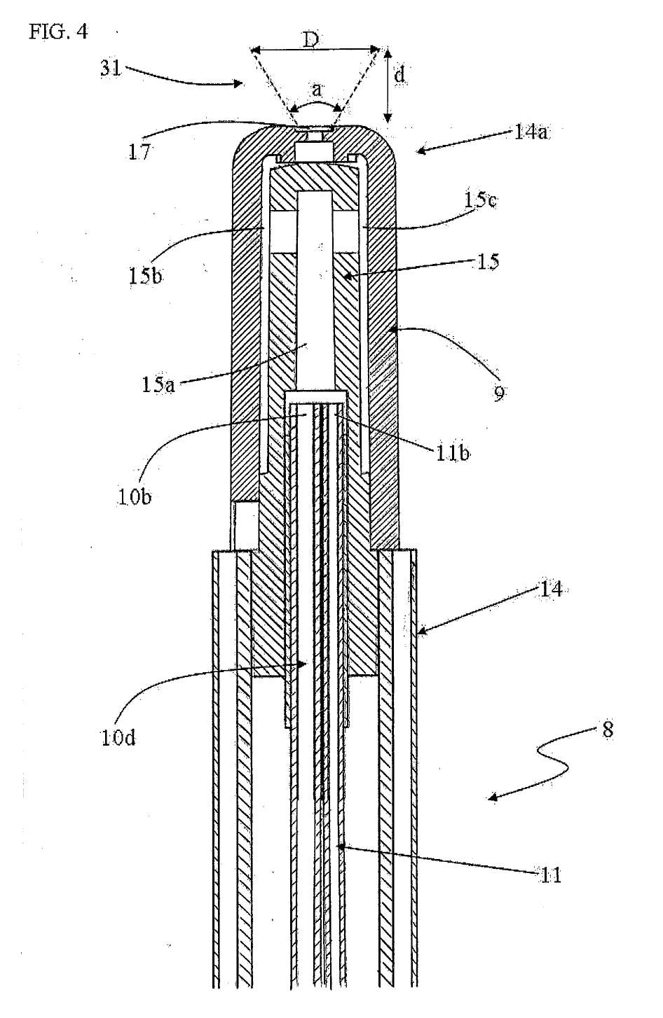

FIGS. 2, 3 and 4 illustrate a lateral view of different portions of the dispenser of FIG. 1.

With reference to the figures, a dispenser for adhesive Newtonian fluid has been indicated with the number 1. In particular, the dispenser 1 finds application in the medical sector, preferably in open or laparoscopic surgery.

Preferably, the dispenser 1 is portable. For example, the dispenser 1 is employed as a single-user instrument for sanitary reasons.

In this context, the adhesive Newtonian fluid consists of a biologically compatible adhesive or glue. In particular, the adhesive Newtonian fluid comprises a bioabsorbable component. For example, the adhesive fluid comprises an antibacterial agent. Said fluid can comprise a free radical stabilizer (e.g. hydroquinone, butylated hydroxyanisole or butylated hydroxytoluene) or an anionic stabilizer (e.g. sulfur dioxide or carbonic acid).

Preferably, the adhesive fluid is made up of a medical cyanoacrylate monomer. Alternatively, said fluid comprises a cyanoacrylate and a co-monomer. Cyanoacrylates have good haemostatic and adhesive properties and, once solidified, they create an effective antiseptic barrier against the most common infective and pathogenic agents in surgical interventions.

In particular, the adhesive fluid used is made up of a monomer capable of polymerizing on contact with a moist surface, such as a body tissue. For example, the adhesive fluid has the following structural formula:

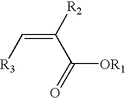

where R₁ is alkyl or alkoxy alkyl or aryl, R₂ is an electron withdrawing group such as CN, CO₂H, Cl, Br or F, while R₃ is alkyl or alkoxy alkyl or aryl.

Preferably, the adhesive fluid comprises a rheological modifier or a plasticizer. Preferably, the adhesive fluid comprises additive agents capable of improving biocompatibility by eliminating or rendering harmless toxic products (e.g. formaldehyde) coming from decomposition.

The dispenser 1 has a first reservoir 2 for the adhesive Newtonian fluid, provided with a first aperture 4 for the egress of the fluid. In the embodiment described herein, the first reservoir 2 is made up of at least one syringe 5 containing the adhesive fluid. The outlet spout of the syringe 5 defines the first aperture 4.

The dispenser 1 is also provided with an inlet 27 for a propellant gas. In particular, the propellant gas is biocompatible and approved for medical use. Preferably, the propellant gas is HFC134/a (1,1,1,2 tetrafluoroethane), that means a non-toxic, biocompatible, non-flammable compound.

Preferably, the dispenser 1 comprises a second reservoir 3 for the propellant gas connected to the inlet 27 of the gas itself. In particular, the second reservoir 3 is provided with a second aperture 6 for the egress of the propellant gas and a duct 29 for guiding the propellant gas so as to connect the second aperture 6 and the inlet 27.

Preferably, said second reservoir 3 is made up of a cylinder 7 containing the gas. Preferably, the cylinder 7 is made of steel or aluminium. In particular, the inner walls of the cylinder 7 are coated with an inert material, such as epoxy resin. The gas present in the cylinder 7 is under pressure, thus the cylinder 7 must be protected from sources of heat.

The dispenser 1 is provided with means 28 for mixing the adhesive fluid coming out from the first aperture 4 and the propellant gas coming from the inlet 27 in order to obtain a mixture deliverable on a surface (e.g. a body tissue).

Innovatively, the means 28 for mixing are shaped in such a way that the mixing of the adhesive fluid and the propellant gas takes place in proximity to an open end 14a of a cannula or catheter 14 of the dispenser 1.

Advantageously, the mixture is atomized from the open end 14a so as to reach the surface and be deposited on the latter.

The dispenser 1 is provided with means 8 for guiding the adhesive fluid and the propellant gas. Said means 8 for guiding the adhesive fluid and the propellant gas are connected to the first aperture 4 and to the inlet 27 for the gas so as to receive the adhesive fluid and the propellant gas and guide them separately towards a confluence element 9.

Advantageously, the confluence element 9 is positioned in correspondence to the open end 14a.

In the embodiment described herein, the means 8 for guiding the adhesive fluid and the propellant gas comprise at least one first duct 10 for the adhesive fluid and at least one second duct 11 for the propellant gas.

In particular, the first duct 10 has an entrance 10a connected to the first aperture 4 and an outlet 10b connected to the confluence element 9. Preferably, the entrance 10a of the first duct 10 is connected to the first aperture 4 by means of a “luer” coupling. The first duct 10 is made up of an initial tract 10c connected to the entrance 10a and of a final tract 10d connected to the outlet 10b. Preferably, the final tract 10d has a smaller diameter than the initial tract 10c.

Preferably, the first duct 10 is provided with a nonreturn valve 30 to prevent the propellant gas from entering the first reservoir 2.

Preferably, the second duct 11 has an entrance 11a connected to the inlet 27 for the propellant gas and an outlet 11b connected to the confluence element 9.

Advantageously, the duct 29 for guiding the gas and the initial tract 10c of the first duct 10 are disposed in such a way as to converge at a funnel 13. The second duct 11 and the final tract 10d of the first duct 10 branch off from said funnel 13. In particular, the second duct 11 and the final tract 10d of the first duct 10 are substantially parallel.

Preferably, the catheter 14 contains at least a portion of the second duct 11 and at least the final tract 10d of the first duct 10. In particular, the catheter 14 is flexible and has a length of around 33 cm and a diameter of around 5 mm. Preferably, the catheter 14 is made up of plastic material approved for medical use, such as polyethylene, polypropylene or PTFE.

Innovatively, the confluence element 9 comprises a diffusing tip 15 provided with a first channel 15a in which the meeting of the adhesive fluid and the propellant gas takes place, and a second channel 15b and a third channel 15c branching off from said first channel 15a. Advantageously, the second channel 15b and the third channel 15c have a smaller diameter than the first channel 15a in such a way as to increase the pressure of the mixture. In particular, the diffusing tip 15 is provided with a nozzle 17 to which the second channel 15b and the third channel 15c are afferent in such a way as to atomize the mixture.

The dispenser 1 is provided with means 22 for activating the adhesive fluid to produce the egress of the fluid from the first aperture 4 to the means 28 for mixing. Said means 22 for activating the fluid are manual or automatic. For example, the means 22 for activating the fluid comprise a plunger 23 to push the fluid out of the syringe 5 through the first aperture 4.

The dispenser 1 is moreover provided with means 24 for activating the propellant gas to produce the egress of the gas from the second aperture 6 towards the duct 29 for guiding the gas. Said means 24 for activating the gas are manual or automatic. In particular, in the embodiment described herein, the means 24 for activating the gas comprise two complementary half-shells 25 inside which the second reservoir 3 is housed. In the embodiment presented herein, the cylinder 7 is housed between a lower half-shell 25a and an upper half-shell 25b. Preferably, the half-shells 25 are made up of plastic material, e.g. ABS (acrylonitrile-butadiene-styrene). Said half-shells 25 are displaceable from a position of rest in which they are spaced apart from each other, to an operative position where they are brought together so as to latch onto each other.

Preferably, on the outside of the upper half-shell 25b there is a housing 12 for the syringe 5.

Preferably, the means 24 for activating the gas comprise a tap 26 operatively active on the guiding duct 29 for the propellant gas. In particular, said tap 26 is displaceable from a closure configuration in which it prevents the propellant gas from continuing towards the inlet 27 for the gas, to a plurality of opening configurations in which it allows the propellant gas to reach the inlet 27 in pre-established quantities.

Alternatively, in the absence of the tap 26, the gas coming out of the second aperture 6 travels continuously toward the inlet 27 for the gas.

The functioning of the dispenser for adhesive Newtonian fluid, according to the present invention, is described below.

The dispenser 1 is gripped with one hand by a user. Preferably, the dispenser 1 is taken hold of by the half-shells 25 in such a way that the cylinder 7 containing the propellant gas is in a lower position than the syringe 5 and the catheter 14 is substantially horizontal. Preferably, the tip 15 is situated at a distance of between 2 cm and 5 cm from the surface to be atomized onto.

With his/her hand engaged, the user brings the two half-shells 25 from the position of rest to the operative position, thereby causing them to latch together. In this way, the gas flows out from the second aperture 6 toward the guiding duct 29.

In the absence of the tap 26, the gas then continues toward the inlet 27 for the gas. In the presence of the tap 26, the user turns the tap 26 in such a way as to bring it from the closure configuration to one of the opening configurations so as to enable a pre-established quantity of gas to reach the inlet 27. Said pre-established quantity of gas thus arrives in the second duct 11.

The user then presses on the plunger 23 of the syringe 5 in such a way as to push the fluid out of the first aperture 4 toward the initial tract 10c of the first duct 10. Said fluid thus arrives at the final tract 10d of the first duct 10.

The gas coming from the second duct 11 and the fluid coming from the final tract 10d of the first duct 10 are mixed inside the diffusing tip 15 and atomized through the nozzle 17.

Depending on the pressure exerted on the plunger 23, the opening configuration of the tap 26 and the distance d of the nozzle 17 from the delivery surface, the atomization parameters will vary. In particular, the delivered fluid defines an atomization profile 31 having substantially a truncated cone shape. The opening of said atomization profile 31 is quantifiable by means of an opening angle a or an atomization diameter D. The number of molecules of delivered fluid present within the atomization profile 31 defines a concentration Q of atomization. For example, by placing the nozzle 17 at a distance d from the surface equal to around 5 cm, in optimal conditions an atomization diameter D equal to around 6 cm and an opening angle a of around 60° will be obtained.

Preferably, once the egress of the fluid from the first aperture 4 ceases (which occurs almost instantaneously when the pressure on the plunger 23 is interrupted), it is necessary to wait a few seconds before bringing the tap 26 back into the closure configuration. This allows the nozzle 17 to be cleaned by the passage of the propellant gas.

The characteristics of the dispenser for adhesive Newtonian fluid, according to the present invention, emerge clearly from the description provided, as do the advantages thereof.

In particular, thanks to the configuration of the diffusing tip, the propellant gas and the adhesive fluid are uniformly mixed and premature polymerization in the nozzle is prevented.

Furthermore, the three channels of the diffusing tip enable the fluid to be atomized, i.e. to be delivered homogeneously across space and thus form a thin film on the surface concerned. Moreover, the second and the third channel of the diffusing tip, having a smaller diameter than the first channel, serve to reduce the contamination of the atomized fluid by airborne dusts.

Furthermore, thanks to the fact that the gas continues to come out for a few seconds after the egress of fluid from the first reservoir ceases, cleaning of the diffusing tip is assured, thus preventing the occurrence of undesired clogging.

Moreover, since the delivery of the fluid takes place at the open end of the catheter, the times of response to the dispensing command (i.e. from the instant at which the plunger is pressed) are practically immediate, allowing the user full control of the quantity of fluid to be delivered.

Furthermore, thanks to the adjustment of the tap and the possibility of varying the distance d between the nozzle and the delivery surface, it is possible to always keep both the quantity of fluid to be delivered (i.e. the concentration Q of atomization) and the area covered (i.e. the opening angle a and the atomization diameter D) under control.

Moreover, the dispenser presents a very simple structure, since the adhesive fluid is contained in the syringe and the propellant gas is in the cylinder. The housing of the syringe above the cylinder also contributes to rendering use of the dispenser easy and ergonomic.

Furthermore, the dispenser is easy to handle thanks to the presence of the two half-shells containing the cylinder and the use of a simple plunger to activate the egress of the fluid. The presence of the catheter also contributes to rendering the dispenser easier to handle, in addition to performing an important protective function for the means for guiding the fluid and the gas.

Finally, since the delivery of the fluid takes place by atomization, the proposed dispenser can be used in open surgery and laparoscopic operations.

1. Dispenser for adhesive Newtonian fluid comprising:
a first reservoir for the adhesive fluid, provided with a first aperture for the egress of said adhesive fluid;
an inlet for a propellant gas;
means for mixing the adhesive fluid coming out from the first aperture and the propellant gas coming from said inlet in order to obtain a mixture deliverable on a surface,
characterised in that said means for mixing are shaped in such a way that the mixing of the adhesive fluid and the propellant gas takes place in proximity to an open end of a cannula or catheter of the dispenser, said mixture being atomized from said open end onto the surface.

2. Dispenser according to claim 1, characterised in that it comprises means for activating the adhesive fluid to produce the egress of said adhesive fluid from the first aperture to the means for mixing.

3. Dispenser according to claim 2, wherein the first reservoir is made up of at least one syringe containing the adhesive fluid, and the means for activating the adhesive fluid comprise a plunger to push the adhesive fluid out of said at least one syringe, the outlet spout of the syringe defining the first aperture.

4. Dispenser according to claim 1, characterised in that it comprises a second reservoir for the propellant gas, connected to said inlet for the propellant gas.

5. Dispenser according to claim 4, characterised in that it comprises means for activating the propellant gas to produce the egress of the gas from a second aperture of the second reservoir towards a duct for guiding said propellant gas.

6. Dispenser according to claim 5, wherein the means for activating the gas comprise a tap operatively active on the duct for guiding the gas and displaceable from a closure configuration in which it prevents the propellant gas from continuing towards the inlet for the gas, to a plurality of opening configurations in which it allows the propellant gas to reach said inlet in pre-established quantities.

7. Dispenser according to claim 5, wherein the means for activating the gas comprise two complementary half-shells inside which the second reservoir is housed, said half-shells being displaceable from a position of rest in which they are spaced apart from each other, to an operative position where they are brought together so as to latch onto each other.

8. Dispenser according to claim 1, characterised in that it comprises means for guiding the adhesive fluid and the propellant gas connected to the first aperture and to the inlet for the gas so as to receive the adhesive fluid and the propellant gas and guide them separately towards a confluence element positioned in correspondence to said open end.

9. Dispenser according to claim 8, wherein the means for guiding the adhesive fluid and the propellant gas comprise at least one first duct for the adhesive fluid and at least one second duct for the propellant gas, said at least one first duct having an entrance connected to the first aperture and an outlet connected to the confluence element and said at least one second duct having an entrance connected to the inlet for the propellant gas and an outlet connected to the confluence element.

10. Dispenser according to claim 9, wherein said first duct is made up of an initial tract connected to the entrance and of a final tract connected to the outlet, the final tract having a smaller diameter than the initial tract.

11. Dispenser according to claim 10, wherein the catheter contains at least a portion of the second duct and at least the final tract of the first duct.

12. Dispenser according to claim 9, wherein the first duct is provided with a nonreturn valve for preventing the propellant gas from entering the first reservoir.

13. Dispenser according to claim 8, wherein the confluence element comprises a diffusing tip provided with a first channel in which the meeting of the adhesive fluid and the propellant gas takes place, and a second channel and a third channel branching off from said first channel and having smaller diameter than said first channel in such a way as to increase the pressure of the mixture.

14. Dispenser according to claim 13, wherein the diffusing tip is provided with a nozzle to which the second channel and the third channel are afferent in such a way as to atomize the mixture.

15. Dispenser according to claim 1, wherein the adhesive fluid is made up of a medical cyanoacrylate monomer.

16. Dispenser according to claim 1, wherein the adhesive fluid is made up of a monomer capable of polymerizing on contact with the surface.

17. Dispenser according to claim 1, wherein the adhesive fluid comprises a rheological modifier or a plasticizer.

18. Dispenser according to claim 1, wherein the adhesive fluid is made up of a bioabsorbable component.

19. Dispenser according to claim 1, wherein the adhesive fluid comprises an antibacterial agent.

20. Dispenser according to claim 1, wherein the adhesive fluid has the following structural formula: 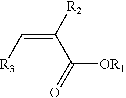
where R₁ is alkyl or alkoxy alkyl or aryl, R₂ is an electron withdrawing group such as CN, CO₂H, Cl, Br or F, while R₃ is alkyl or alkoxy alkyl or aryl.

21. Dispenser according to claim 1, wherein the adhesive fluid comprises a free radical stabilizer or an anionic stabilizer.

22. Dispenser according to claim 1, wherein the adhesive fluid comprises additive agents capable of improving biocompatibility by eliminating or rendering harmless toxic products coming from decomposition.

23. Dispenser according to claim 1, wherein the propellant gas is biocompatible and approved for medical use.

24. Dispenser according to claim 1, wherein the catheter is made up of plastic material approved for medical use.

25. Dispenser according to claim 1, characterised in that it is portable.

26. Dispenser according to the claim 2, wherein the means for activating the adhesive fluid are manual or automatic.

27. Dispenser according to claim 5, wherein the means for activating the gas are manual or automatic.
